(19) United States
(12) Patent Application Publication
(10) Pub. No.: US 2008/0086214 A1
(43) Pub. Date: Apr. 10, 2008

(54) Medical device having a sleeve valve with bioactive agent

(75) Inventors: David M. Hardin, Winston-Salem, NC (US); Kulwinder S. Dua, Brookfield, WI (US); Gregory J. Skerven, Kernersville, NC (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Wilson-Cook Medical Inc., Winston-Salem, NC (US)

(21) Appl. No.: 11/810,307

(22) Filed: Jun. 5, 2007

Related U.S. Application Data
(63) Continuation-in-part of application No. 11/341,970, filed on Jan. 27, 2006.
Application No. 11/341,970 is a continuation-in-part of application No. 10/208,736, filed on Jul. 29, 2002, now Pat. No. 7,118,600.
Application No. 10/208,736 is a continuation-in-part of application No. 09/876,520, filed on Jun. 7, 2001, now Pat. No. 6,746,489.
Application No. 10/208,736 is a continuation-in-part of application No. 09/386,173, filed on Aug. 31, 1999, now Pat. No. 6,302,917.
(60) Provisional application No. 60/211,753, filed on Jun. 14, 2000.
(60) Provisional application No. 60/098,542, filed on Aug. 31, 1998.
(60) Provisional application No. 60/648,744, filed on Jan. 31, 2005.
(60) Provisional application No. 60/811,647, filed on Jun. 7, 2006.

(51) Int. Cl. A61F 2/04

(52) U.S. Cl. 623/23.7

(57) ABSTRACT

Medical devices for implantation in a body vessel are provided. A medical device can be configured as a drainage stent adapted for placement in a bodily passageway. The drainage stent preferably includes a drainage lumen extending longitudinally through the drainage stent, and a sleeve defining a collapsible lumen in fluid flow communication with the drainage lumen. The sleeve may function as a one-way valve and preferably includes a biodeposition-reducing bioactive agent, such as an antibiotic or antimicrobial agent. The medical device may be configured as a biliary or pancreatic stent.

### RELATED APPLICATIONS

This application claims the benefit of U.S. provisional patent application 60/811,647, filed Jun. 7, 2006; this application is also a continuation-in-part of U.S. patent application Ser. No. 11/341,970, filed Jan. 27, 2006, which is a continuation-in-part of U.S. patent application Ser. No. 10/208,736, filed Jul. 29, 2002 and issued as U.S. Pat. No. 7,118,600, which is a continuation-in-part of U.S. patent application Ser. No. 09/876,520, filed Jun. 7, 2001, which issued as U.S. Pat. No. 6,746,489, which claims priority to U.S. Provisional Application Ser. No. 60/211,753, filed Jun. 14, 2000, and is a continuation-in-part of U.S. patent application Ser. No. 09/386,173, filed Aug. 31, 1999, which issued as U.S. Pat. No. 6,302,917, and which claims priority to U.S. Provisional Application Ser. No. 60/098,542, filed Aug. 31, 1998. This application also claims priority to U.S. Provisional Application Ser. Nos. 60/309,107, filed Jul. 31, 2001 and 60/648,744, filed Jan. 31, 2005. All of the above-referenced patents and patent applications are hereby incorporated by reference in their entirety.

### TECHNICAL FIELD

The present invention relates to implantable medical devices. More particularly, the invention relates to drainage stents comprising a bioactive, including drainage stents adapted for use in the biliary tract.

### BACKGROUND

Endoluminal medical devices can be implanted to treat various conditions. For example, a biliary stent can be implanted within a biliary duct to treat conditions associated with compromised drainage of the biliary tree, such as obstructive jaundice. Implanted biliary stents can provide for the palliation of malignant biliary obstruction, particularly when surgical cure is not possible. Biliary stenting treatment approaches can also be used to provide short-term treatment of conditions such as biliary fistulae or giant common duct stones. Long term implantation of biliary stents can be used to treat chronic conditions such as postoperative biliary stricture, primary sclerosing cholangitis and chronic pancreatitis.

Biliary stents may be configured as a tubular structure housing a drainage lumen. The biliary stent may be sufficiently flexible to be advanced on a delivery catheter or through an endoscope along a path that may include sharp bends, before being placed in a bile duct. The biliary stent may also be sufficiently strong to resist collapse and to maintain an open drainage lumen through which digestive liquids can flow into the digestive tract. The biliary stent also should maintain its intended position within the bile duct without migrating from that position.

Once implanted, biliary stents can become occluded within a bile duct, as an encrustation of amorphous biological material and bacteria (“sludge”) accumulate on the interior surface of the stent, gradually obstructing the lumen of the stent. Biliary sludge is an amorphous substance often containing crystals of calcium bilirubinate and calcium palimitate, along with significant quantities of various proteins and bacteria. Sludge can deposit rapidly upon implantation in the presence of bacteria. For example, bacteria can adhere to plastic stent surfaces with pili or through production of a mucopolysaccharide coating. Bacterial adhesion to the wall of a drainage lumen can result in occlusion of the drainage stent, as the bacteria multiply within a glycocalyx matrix of the sludge to form a biofilm over the sludge within the drainage lumen of an implanted drainage stent. The biofilm can provide a physical barrier protecting encased bacteria within the sludge from contact with host white blood cells and antibodies, and diminishing the penetration of antibiotics into the stent sludge. With time, an implanted biliary stent can become blocked, thereby restricting or blocking bile flow through the drainage stent. As a result, a patient can develop symptoms of recurrent biliary obstruction due to restricted or blocked bile flow through an implanted biliary stent, which can be complicated by cholangitis and sepsis. Often, such conditions are treated by antibiotics and/or endoscopic replacement of an obstructed biliary stent.

In addition to clogging, another post-implantation challenge after the implantation of a biliary stent may be reducing or preventing undesired retrograde fluid flow through the drainage lumen. Retrograde fluid flow through a biliary stent may create a risk of migration of bacteria into the drainage lumen, which could lead to infection or obstruction of the drainage lumen.

Therefore, there exists a need for an endoluminal medical device, such as a drainage stent, that desirably reduces retrograde flow through a body vessel while simultaneously preventing or reducing bacteria, biofilm and sludge deposition inside the drainage lumen of implantable medical device. Promising approaches for preventing biofilm and sludge deposition have involved systemic administration of antibiotics, such as fluoroquinolone agents, that achieve high concentrations in bile and are effective against enteric Gram-negative bacteria. However, systemic treatment approaches may not allow penetration of the antibiotic agent through the glycocalyx matrix of biofilm that can insulate bacteria from contact with the antibiotic.

What is needed is a medical device having a drainage lumen adapted to regulate antegrade and/or retrograde flow through the drainage lumen in response to the fluid flow within a body vessel, while delivering one or more bioactive agents that prevent or mitigate the deposition of bacteria or other material that can lead to blockage of a drainage lumen in the medical device.

### SUMMARY

The present disclosure relates to endoluminal medical devices, such as drainage stents, comprising a drainage lumen with a valve means for regulating fluid flow through the drainage lumen, and a releasable biodeposition-reducing bioactive agent. The drainage lumen is defined by an interior surface of the drainage stent, and may extend longitudinally from an inlet to an outlet along the axis of the drainage stent. The valve means is preferably configured as a sleeve in communication with the drainage lumen. A portion of the medical device contacting the fluid flow can contain a releasable biodeposition-reducing bioactive agent. Preferably, the sleeve contains the biodeposition-reducing bioactive agent, although the biodeposition-reducing bioactive agent can also be positioned on the surface of the drainage lumen.

In one embodiment, the endoluminal medical device is a drainage stent comprising a collapsible sleeve comprising a releasable biodeposition-reducing bioactive agent attached to the outlet of a tubular drainage stent, such as a biliary stent, to advantageously prevent reflux of intestinal contents and the associated bacteria into the drainage lumen of the stent. The biodeposition-reducing bioactive agent may be an antibiotic or antimicrobial agent, to prevent formation of biofilm within the drainage lumen of the medical device, which can lead to occlusion of the drainage lumen. The sleeve can define a collapsible lumen that is preferably positioned in fluid flow communication with the drainage lumen of a biliary stent. The collapsible lumen of the sleeve can be positioned within the drainage lumen of a drainage stent or may extend longitudinally from the drainage lumen of the drainage stent.

Preferably, one end of the sleeve material circumferentially encloses the outlet end of a biliary stent. The sleeve material is preferably configured as a tube of flexible material, and may have any suitable thickness. Advantageously, the sleeve is long enough to permit shortening the sleeve length to accommodate variation in individual anatomy. Depending on the anatomical size of the human or veterinary patient, the sleeve can extend from the outlet end of the tubular drainage stent for any suitable length, for example up to about 20 cm (about 7.9 inches), preferably in a range of 5 to 15 cm (about 2.0 inches to 5.9 inches), and most preferably approximately 10 cm (about 3.9 inches) in a human patient or 8 cm (3.1 inches) in a veterinary patient. The sleeve material can be formed from any biocompatible material that is flexible and acid resistant, preferably expanded-polytetrafluoroethylene (“ePTFE”). The sleeve can also be formed from polyurethane, silicone, or polyamides (including a nylon material).

The sleeve may function as a valve by collapsing or inverting to block fluid flow in a retrograde direction, into the outlet of a drainage stent. The sleeve may be configured as a flexible tube defining a collapsible lumen, and having an exterior surface. Fluid flow in the antegrade direction may provide a first pressure against the collapsible lumen of the sleeve in the antegrade direction, effective to expand the collapsible lumen of the sleeve and permit fluid to flow through the sleeve from the outlet of the drainage stent. However, fluid flow in the retrograde direction may exert a second pressure against the sleeve effective to collapse the sleeve. The sleeve may collapse when the second pressure is greater than the first pressure, thereby blocking fluid flow into the drainage lumen of the drainage stent. The pressure needed to collapse or invert the sleeve can be a function of the sleeve material, thickness and length measured from the distal end of a tube of a drainage stent. The thickness of the sleeve can vary as a function of distance from the outlet of the biliary stent. Desirably, the sleeve material is thicker at the portion attached to the drainage stent, and progressively thinner moving away from the drainage stent outlet. For example, the sleeve may desirably have a thickness of about 0.0050-inch (about 0.0127 mm) through about 0.0080-inch (about 0.0203 mm) at the portion attached to the drainage stent outlet, but a decreasing thickness in a range of about 0.0040-inch (about 0.1016 mm) to about 0.0015-inch (about 0.0381 mm), preferably approximately 0.0020-inch (about 0.0508 mm), at the sleeve portion distal to the portion attached to a drainage stent outlet.

A drainage stent configured as a biliary stent is desirably placed in the biliary tree for maintaining patency of the bile or pancreatic duct and the Papilla of Vater. Preferably, the biliary stent is positioned so that the sleeve can extend down into the duodenum to provide a one-way valve for the flow of bile. When bile is not being secreted, the sleeve advantageously collapses to prevent backflow of material from the duodenum, which might otherwise occur in a biliary stent without a valve means. Alternatively, the sleeve may be located completely within the lumen of the drainage stent with one end of the sleeve being bonded or otherwise attached to the interior wall of the biliary stent. Alternatively, the drainage stent can also be configured for placement in the ureters or urethra, and can include a sleeve extending from one end of the drainage conduit to permit urine flow and prevent retrograde flow or pathogen migration toward the kidneys or bladder.

In yet another aspect of the present invention, a method of treating a subject comprises implanting a medical device at a point of treatment, such as within a biliary duct, wherein the medical device comprises a tubular member and a sleeve.

### BRIEF DESCRIPTION OF THE DRAWINGS

FIG. 1 is a side view of a first biliary stent embodiment;

FIG. 2 is a longitudinal cross sectional view of a portion of the biliary stent shown in FIG. 1.

FIG. 3 depicts a side view of one end of a valved prosthesis that includes a pigtail configuration.

FIG. 4 depicts a laterally sectioned view of a valved prosthesis in which the sleeve is affixed with the lumen.

FIG. 5 depicts a two piece mandril that is used to apply the sleeve material to the prosthesis of FIG. 3.

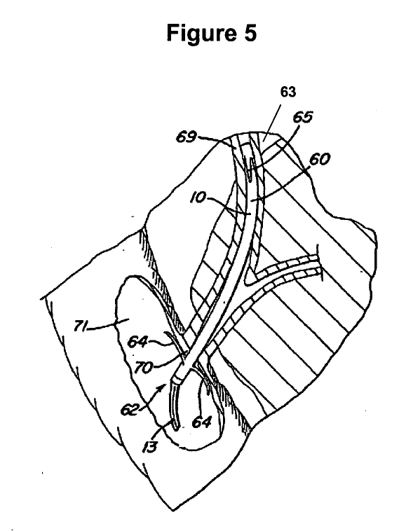

FIG. 6 depicts the anti-reflux esophageal prosthesis of FIG. 3 in a collapsed state in a delivery catheter.

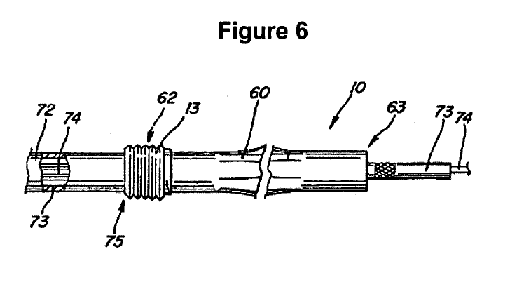

### DETAILED DESCRIPTION

The following detailed description and appended drawings describe and illustrate various exemplary embodiments of the invention. The description and drawings serve to enable one skilled in the art to make and use the invention, and are not intended to limit the scope of the invention in any manner.

The invention provides medical devices for implantation in a body vessel, methods of making the medical devices, and methods of treatment that utilize the medical devices.

As used herein the terms “comprise(s),” “include(s),” “having,” “has,” “contain(s),” and variants thereof, are intended to be open-ended transitional phrases, terms, or words that do not preclude the possibility of additional acts or structure.

The term “effective amount” refers to an amount of an active ingredient sufficient to achieve a desired affect without causing an undesirable side effect. In some cases, it may be necessary to achieve a balance between obtaining a desired effect and limiting the severity of an undesired effect. It will be appreciated that the amount of active ingredient used will vary depending upon the type of active ingredient and the intended use of the composition of the present invention.

As used herein, the term “body vessel” means any body passage that conducts fluid, including but not limited to biliary ducts, ureteral passages, esophagus, and blood vessels such as those of the human vasculature system.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

As used herein, the term “biodeposition-reducing bioactive agent” refers to a material that reduces the rate of biodeposition within the lumen of a drainage stent. Biodeposition can include the deposition of components of the biofilm or glycocalyx matrix on the interior surface of the drainage stent, such as calcium bilirubinate, calcium palimitate, proteins and bacteria. Biodeposition-reducing bioactive agents are preferably antibiotic or antimicrobial agents, although any other suitable materials can be used.

As used herein, “endolumenally,” “intraluminally” or “transluminal” all refer synonymously to implantation placement by procedures wherein the medical device is advanced within and through the lumen of a body vessel from a remote location to a target site within the body vessel. Endolumenal delivery includes implantation in a biliary duct from an endoscope or catheter.

A “biocompatible” material is a material that is compatible with living tissue or a living system by being medically appropriate for a given treatment. Preferably, a biocompatible material does not induce an undesirable level of toxicity, injury or immunological rejection upon implantation for a desired therapeutic outcome. Biocompatibility tests may include tests and standards set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.”

The invention relates to medical devices for implantation in a body vessel. More specifically, various embodiments of the invention relate to a medical device comprising a sleeve formed from a flexible material, the sleeve attached to a drainage stent and having a lumen extending longitudinally there through and communicating with a drainage lumen extending through the drainage stent. The sleeve desirably comprises ePTFE containing one or more biodeposition-reducing bioactive agents. The sleeve preferably defines a collapsible lumen in communication with the outlet of a biliary stent. The sleeve may be configured to open in response to a fluid flow out of the outlet of the biliary stent. The fluid flow from the biliary stent may apply a first pressure on the sleeve in a first direction, opening the sleeve lumen to permit the fluid flow to pass the fluid through the sleeve lumen. However, movement of the sleeve in response to fluid flow in the opposite direction, toward the outlet of the biliary stent, can collapse the sleeve lumen to at least substantially close the lumen of the sleeve, and block retrograde flow into the biliary stent outlet. The sleeve may be configured to collapse in response to either a fluid applying a second pressure in a second direction or the absence of the fluid applying a first pressure in a first direction.

Medical Device Configurations

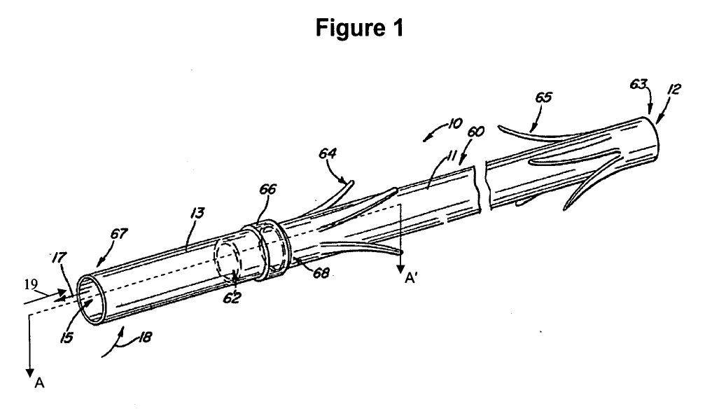

FIG. 1 shows a medical device 10 comprising a tubular member 11 that can be configured as a tubular drainage stent 60 having a drainage lumen 12 extending longitudinally from an inlet 63 to an outlet 62 for drainage of fluid through a body passage such as a duct, vessel, organ, and the like. Unless otherwise indicated, the terms “inlet” and “outlet” refer to the antegrade direction of fluid flow through a medical device as being into the medical device through the inlet and exiting the medical device from the outlet, but do not preclude reverse (retrograde) fluid flow in the opposite direction, or bidirectional flow in both antegrade and retrograde directions. Typically, medical devices are implanted to permit fluid to flow through the medical device in substantially the antegrade direction.

The drainage lumen 12 is defined by an interior surface of the medical device 10. The inlet 63 is adapted to receive the fluid or other material that is moving under a first, antegrade direction 17 at a first pressure. The collapsible sleeve 13 is preferably in fluid flow communication with the drainage lumen 12, meaning that fluid flow may pass through the drainage lumen 12 before, during or after passing through the collapsible sleeve 13. The outlet 62 of the tubular drainage stent 60 may be circumferentially enclosed by the sleeve 13, or the sleeve 13 may be positioned within the drainage lumen 12. The sleeve 13 may be a tube of flexible material extending from a first end 67 to a second end 68. The second end 68 is preferably positioned around the outlet 62 of the drainage stent 60, for example by a retaining ring 66. The sleeve 13 may be adapted to function as a collapsible one-way valve to prevent or reduce fluid flow in a retrograde direction 19 into the outlet 62 and through the drainage stent 60.

Preferably, the medical device comprises a means for anchoring the device within a body passage. The means for anchoring the biliary stent may include flaps extending from the exterior surface of the tubular member 11. The number, size and orientation of anchoring flaps can be modified to accommodate the migration-preventing requirements of the particular medical device to be implanted, the site of implantation and the desired function of the device. For example, the drainage stent 60 comprises an outlet array 64 and an inlet array 65 of radially extending flaps extending from the exterior surface of the drainage stent 60, proximate the outlet 62 and the inlet 63, respectively. The outlet array 64 and inlet array 65 of flaps can have any suitable number, size and configuration of flaps selected to anchor drainage stent 60 within a biliary duct. For example, the outlet array 64 and the inlet array 65 may comprise one row of four flaps each. The arrays of anchoring flaps 64, 65 can be formed by slicing small longitudinal sections in the distal or proximate ends of the tubular member 11 and orienting the sliced sections radially. Preferably, the slice incisions are made in the exterior surface of the tubular member 11 in a shallow manner so as to not create holes in the drainage stent 60. Alternatively, the drainage stent 60 may also include an anchoring means, such barbs, pigtail loops, etc. positioned proximate the outlet 62 and/or the inlet 63.

The sleeve 13 is preferably configured to act as a one-way valve permitting substantially uni-directional fluid flow through the drainage lumen 12 of the drainage stent 60. Referring to FIG. 1, the sleeve 13 is shown in an open configuration permitting fluid or material to pass through the sleeve 13 in the antegrade direction 17, exerting a first radial pressure directed outward against the sleeve 13. The sleeve 13 is preferably highly flexible and readily collapsible. The sleeve 13 may be configured to function as a one-way valve by selecting sleeve material that is sufficiently flexible to collapse the sleeve lumen in the absence of sufficient fluid flow in an antegrade direction 17, thereby permitting opposable portions of the sleeve material adhere to one another, particularly if wet. Fluid pressure in the retrograde direction 19 or in a second direction 18 may also facilitate closure of the sleeve 13 across the outlet 62. The sleeve 13 may assume a closed configuration, blocking the outlet 62, in the absence of sufficient fluid flow from the outlet 62 in an antegrade direction 17. The sleeve 13 may also assume a closed configuration when fluid (air or liquid) flow applies a second pressure in a second direction 18 to at least substantially close the sleeve lumen 15. The sleeve lumen 15 at the first end 67 may collapse shut when the fluid flow in the antegrade direction 17 has ceased or lessened such that the second fluid pressure in the second direction 18 occurring in the environment into which the fluid is drained becomes greater than the first pressure of the fluid flow in the antegrade direction 17. In the closed configuration, the sleeve 13 may occlude the outlet 62. When closed, the sleeve 13 may greatly reduce migration of fluids, materials, or pathogens into the outlet 62 in the retrograde direction 19, and into the drainage lumen 12 of the drainage stent 60.

Preferably, the sleeve 13 is mounted around outlet 62 of the drainage stent 60 and extends longitudinally therefrom. The range of sleeve thickness for the illustrative embodiment in FIG. 1 may be about 0.0010 to 0.0200 inch (about 0.0254 mm to 0.5080 mm), with a more preferred thickness of about 0.0015 (about 0.0381 mm) to 0.0080 inch (about 0.0203 mm). The thickness of the sleeve can vary as a function of distance from the outlet of the biliary stent. Desirably, the sleeve material is thicker proximate to the portion of the sleeve attached to the second end 68 of the tubular drainage stent 60, and progressively thinner moving toward the sleeve end 67 distal to the attachment portion. Preferably, the thickness of the sleeve material disposed around the tubular drainage stent can be about 0.0050 inch (about 0.0127 mm) to 0.0080 inch (about 0.0203 mm), and most preferably approximately 0.0060 inch thick (about 0.1524 mm). The thickness of the sleeve material at first end 67 of the tubular drainage stent 60 of the sleeve typically ranges from 0.0015 inch (about 0.0381 mm) through 0.0040 inch (about 0.1016 mm) and is preferably about 0.0020 inch (about 0.0508 mm) thick. The length of the sleeve material can be individually customized by the physician depending on the anatomy of the patient. Preferably, the length of the sleeve material extending from the distal end of the tubular drainage stent can range from about 0 through 20 cm (about 7.9 inches), preferably 5 to 15 cm (about 2.0 to 5.0 inches), and more preferably about 10 cm (about 3.9 inches).

The sleeve 13 may be made of a biocompatible material that will not substantially degrade in the particular environment of the human body into which it is to be placed. Possible materials include expanded polytetrafluoroethylene (ePTFE), Dacron, PTFE, TFE or polyester fabric, polyurethane, silicone, nylon, polyamides such as other urethanes, or other biocompatible materials. It is important that the sleeve material be selected appropriately. For example, in the illustrative embodiment, the sleeve is typically made of a tubular piece of ePTFE which may be more resistant to the caustic bile than would a sleeve of polyurethane. The ePTFE tube may be extruded into a thin wall tube having sufficient flexibility to collapse and seal against the ingress of fluid, while having sufficient integrity to resist tearing.

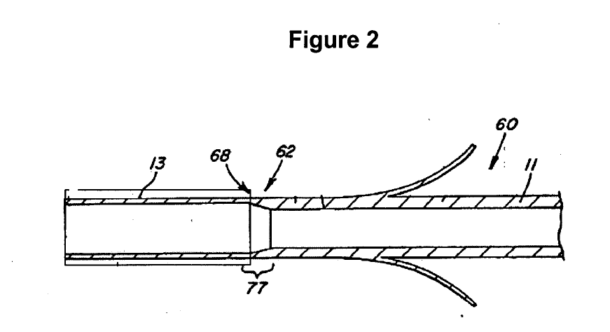

The second end 68 of the sleeve 13 may be attached about the outlet 62 of the drainage stent 60, which can be a ST-2 SOEHENDRA TANNENBAUM® stent, a COTTON-LEUNG® stent or a COTTON-HUIBREGTSE® stent (Cook Endoscopy Inc., Winston-Salem, N.C.), by an attachment means, such as an illustrative crimped metal retaining ring 66. This retaining ring 66 can be made radiopaque to serve as a fluoroscopic marker. Other methods of attachment could include suture binding, selected medical grade adhesives, or thermal bonding, if appropriate for both the sleeve and stent polymers. An alternative method of attaching the sleeve to a tubular drainage stent 60 is depicted in FIG. 2. Rather than attaching a separately extruded or preformed sleeve 13 to the tubular member 11 with the retaining ring 66 (FIG. 1), the wall of the tubular member 11 in FIG. 2 may be thinned out and extended distally from the outlet 62 of the tubular drainage stent 60, such that the sleeve 13 is integral with the tubular member 11. The drainage stent may be made of any suitable material such as polyethylene. A transition zone 77 may exist between the outlet 62 of the tubular drainage stent 60 and the second end 68 of the sleeve 13, beyond which the sleeve 13 becomes sufficiently thin to collapse into a closed position in the absence of antegrade flow 17, such as bile fluid flow.

The drainage stent 60 may be configured as an elongate, closed tubular member housing a drainage lumen 12 providing a fluid drainage conduit adapted to be placed within a bodily passage, such as the bile duct, pancreatic duct, urethra, etc. to facilitate the flow of fluids therethrough. Alternatively, the drainage stent 60 may be configured as a tubular drainage catheter. A drainage stent 60 is commonly implanted either to establish or maintain patency of the bodily passage or to drain an organ or fluid source, such as the liver, gall bladder or urinary bladder. The drainage stent 60 is desirably formed from plastic or metal, and is typically non-expanding.

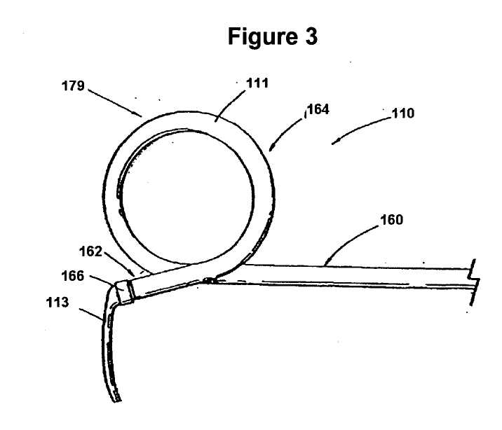

For example, FIG. 3 depicts a second medical device 110 comprising a tubular member 160 that is configured for placement as conduit (e.g., a shunt, stent or drainage catheter) in the urinary system, such as within the ureter between the kidney and the bladder. The sleeve 113 is attached to the first end 162 of the tubular member 160, which includes a first retention means 164 that comprises a curled portion 211 of the tubular member 160 forming a “pigtail” configuration 179. In a ureteral stent, the pigtail 179 would be placed within the bladder to prevent migration of the stent. Optionally, a pigtail configuration 179 can be used to anchor the second end of the stent (not shown), typically within the ureteropelvic junction. The pigtail configuration is exemplary of a large variety of well know pigtail ureteral and urethral stents. The sleeve 113 may be substantially identical to the sleeve 13 described above. The sleeve 113 functions as a one-way valve permitting antegrade fluid flow out of the distal end of the tubular member 160 at the first end 162 and substantially prevents retrograde fluid flow in the opposite direction, into the first end 162. The sleeve 113 is formed from any suitably biocompatible and flexible material, and is desirably collapsible in response to pressure from fluid on the outside of the tubular member 160. Any suitable attachment means 266 is employed to join the sleeve 113 around the first end 162, such as an adhesive or retaining ring. Preferably, the sleeve 113 includes a biodeposition-reducing bioactive agent.

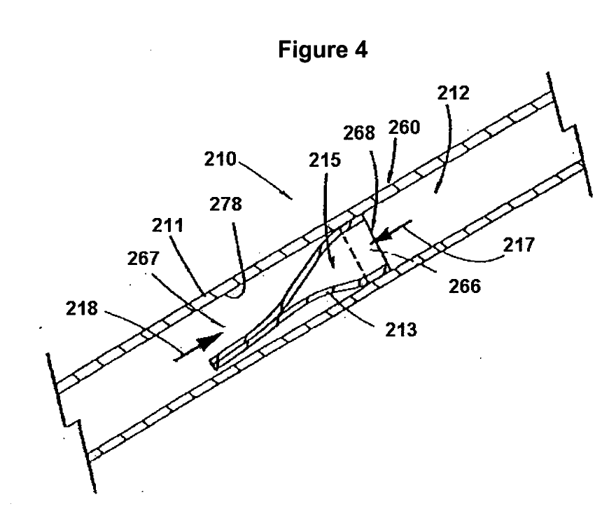

FIG. 4 depicts a portion of another exemplary medical device having a tubular portion 260 in which the first end 268 of the sleeve 213 is affixed completely within the lumen 212 of the tubular portion 260 of a medical device 210. The sleeve 213 is attached to the interior wall 278 of the tubular portion 260 by any suitable attachment means 266, such as thermal bonding, adhesive, or a retaining ring of material securing the sleeve 213 material to the inner wall 278 of the tubular portion 260. In the illustrative embodiment, the sleeve 213 resides completely within the lumen 212 of a tubular portion 260 of a medical device such as a catheter, drainage tube or drainage stent such that the sleeve 213 does not extend beyond the end of the tubular drainage stent 212. This could have particular utility in a urethral stent to prevent migration of pathogenic organism though the stent and into the bladder, while still allowing the flow of urine in the antegrade direction 217. Preferably, the sleeve 213 does not extend out of the urethra and may be located anywhere along the length of a drainage stent or other medical device including a tubular portion 260. Optionally, the external surface 211 of the tubular portion 260 is coated with a bioactive coating, such as an antibacterial agent, analgesic agent and/or a lubricious coating. The sleeve 213 permits fluid flow in an antegrade direction 217 while preventing fluid flow in the opposite retrograde direction 218. Fluid flow in the antegrade direction 217 enters the lumen 215 of the sleeve and forces the sleeve 213 open. Fluid flow in the antegrade direction 217 closes the sleeve 213 and accumulates in a second portion of the lumen 267 of the tubular portion 260 outside the sleeve 213. Preferably, the sleeve 213 includes a biodeposition-reducing bioactive agent.

In another embodiment, the medical device is a medical device comprising: a tubular portion having a passage (e.g., a stent or drainage catheter) extending longitudinally therethrough; and a sleeve disposed around and extending at least partially along said tubular portion, said sleeve extending from an end of said tubular portion and having a lumen extending longitudinally through the sleeve and communicating with said lumen of the tubular portion. The sleeve is preferably configured to collapse in response to a fluid applying a first pressure in a first direction passing the fluid through said lumen of the sleeve, said sleeve collapsing in response to a fluid applying a second pressure in a second direction.

In one embodiment, the medical device includes a tubular member having a passage extending longitudinally therethrough; and a sleeve extending from an end of the tubular member and having a lumen extending longitudinally therethrough and communicating with the passage of the tubular member. The sleeve may be configured to permit the passage of a fluid through the lumen in a first direction in response to the fluid applying a first pressure to the sleeve in the first direction. The sleeve is typically collapsible so as to substantially close the lumen in response to a fluid applying a second pressure to the sleeve in a second direction. The sleeve may also include a proximal portion and a distal portion, and wherein the distal portion comprises a modification with respect to the proximal portion for increasing resistance to being inverted through the tubular stent in response to the second pressure. The sleeve may be normally closed in the absence of the fluid applying the first pressure to the sleeve in the first direction. Optionally, the sleeve may include a proximal portion and a distal portion wherein the distal portion includes an inversion inhibition means for preventing the sleeve from being inverted through the tubular stent in response to the second pressure. The sleeve may optionally include a portion having increased resistance to being inverted through the tubular stent in response to the second pressure; wherein the sleeve may extend through the passage of the tubular member in response to a third pressure that is applied to the sleeve in the second direction, said third pressure being significantly greater than the second pressure; and wherein the sleeve comprises a proximal portion extending from said tubular stent and a distal portion, said distal portion comprising a thickness that is greater than a thickness of said proximal portion for increased resistance to being inverted.

In another embodiment, the medical device may be a drainage stent or catheter for placement in a patient comprising a tubular portion having a passage extending longitudinally therethrough and a sleeve extending from an end of the tubular portion. The sleeve typically defines a lumen extending longitudinally therethrough and communicating with the passage of the tubular portion, the sleeve permitting the passage of a fluid through the lumen of the tubular portion in a first direction in response to the fluid applying a first pressure to the sleeve in the first direction, the sleeve being collapsible so as to substantially close the lumen in response to a fluid applying a second pressure to the sleeve in a second direction. The sleeve may include a portion having increased resistance to being inverted through the tubular stent in response to the second pressure; wherein the sleeve extends through the passage of said tubular portion in response to a third pressure that is applied to the sleeve in the second direction, said third pressure being significantly greater than the second pressure. The sleeve optionally includes a proximal portion extending from said tubular portion of the medical device and a distal portion, said distal portion comprising a material having stiffness that is greater than a stiffness of a material of said proximal portion for increased resistance to being inverted.

Drainage Stent Structure

The drainage stent 60 can be made from any biocompatible material that is resiliently compliant enough to readily conform to the curvature of the duct in which it is to be placed, while having sufficient “hoop” strength to retain its form within the duct. The drainage stent 60 can be formed from any suitable biocompatible material. Preferably, the drainage stent 60 is formed from a thermoformable material such as a polyolefin. One preferred type of material is a metallocene catalyzed polyethylene, polypropylene, polybutylene or copolymers thereof. Preferably, the drainage stent 60 is formed from a biocompatible polyethylene. Other suitable materials for the drainage stent 60 include: vinyl aromatic polymers such as polystyrene; vinyl aromatic copolymers such as styrene-isobutylene copolymers and butadiene-styrene copolymers; ethylenic copolymers such as ethylene vinyl acetate (EVA), ethylene-methacrylic acid and ethylene-acrylic acid copolymers where some of the acid groups have been neutralized with either zinc or sodium ions (commonly known as ionomers); polyacetals; chloropolymers such as polyvinylchloride (PVC); fluoropolymers such as polytetrafluoroethylene (PTFE); polyesters such as polyethyleneterephthalate (PET); polyester-ethers; polyamides such as nylon 6 and nylon 6,6; polyamide ethers; polyethers; elastomers such as elastomeric polyurethanes and polyurethane copolymers; silicones; polycarbonates; and mixtures and block or random copolymers of any of the foregoing. Examples of specific preferred materials for forming the drainage stent include: polyethylene, polyurethane (such as a material commercially available from Dow Corning under the tradename PELLETHANE), silicone rubber (such as a material commercially available from Dow Corning under the tradename SILASTIC), and polyetheretherketone (such as a material commercially available from Victrex under the tradename PEEK). These materials are non-limiting examples of non-biodegradable biocompatible matrix polymers useful for manufacturing the medical devices of the present invention.

A preferred drainage stent 60 structure having a straight configuration (FIG. 1) is the COTTON-LEUNG® (Amsterdam) Biliary Stent (Cook Endoscopy, Winston-Salem, N.C., USA). Alternatively, the drainage stent 60 may have a bent or “pigtail” configuration. Examples of suitable drainage stents 60 having a bent configuration include: MARATHON® Biliary Stents, COTTON-HUIBREGTSE® Biliary Stents, COTTON-LEUNG® (Amsterdam) Stents, GEENEN® Pancreatic Stents, ST-2 SOEHENDRA TANNENBAUM Biliary Stents and JOHLIN® Pancreatic Wedge Stents, all commercially available from Cook Endoscopy Inc. (Winston-Salem, N.C., USA). Examples of suitable drainage stents 60 having a coiled (“pigtail”) inlet and outlet configuration include: Double Pigtail Stent, the ZIMMON® Biliary Stent and the ZIMMON® Pancreatic Stents, all commercially available from Cook Endoscopy Inc. (Winston-Salem, N.C., USA). Other suitable drainage stent configurations are provided in U.S. Pat. Nos. 6,746,489 (Dua et al.) and 6,302,917 (Dua et al.), as well as U.S. patent application Ser. No. 10/827,957, filed Apr. 20, 2004 and published on Oct. 7, 2004 as US 2004/0199262 A1, are incorporated herein by reference in their entirety.

A stent or delivery device may comprise one or more radiopaque materials to facilitate tracking and positioning of the medical device, which may be added in any fabrication method or absorbed into or sprayed onto the surface of part or all of the medical device. For example, referring to FIG. 1, the tubular member 11, or other portion of the drainage stent 60, may be provided with marker bands comprising a radiopaque material at one or both of the outlet 62 and/or the inlet 63. A marker band can provide a means for orienting the stent within a body lumen. The marker band, such as a radiopaque portion of the tubular member, can be identified by remote imaging methods including X-ray, ultrasound, Magnetic Resonance Imaging and the like, or by detecting a signal from or corresponding to the marker. In other embodiments, the delivery device can comprise radiopaque indicia relating to the orientation of the tubular drainage stent within the body vessel.

A marker band may be formed from a suitably radiopaque material. The degree of radiopacity contrast can be altered by altering the content of the radiopaque marker band. Radiopacity may be imparted by covalently binding iodine to the polymer monomeric building blocks of the elements of the implant. Common radiopaque materials include barium sulfate, bismuth subcarbonate, and zirconium dioxide. Other radiopaque elements include: cadmium, tungsten, gold, tantalum, bismuth, platinum, iridium, and rhodium. In one preferred embodiment, iodine may be employed for its radiopacity and antimicrobial properties. Radiopacity is typically determined by fluoroscope or x-ray film. Imagable markers, including radiopaque material, can be incorporated in any portion of a medical device. For example, radiopaque markers can be used to identify a long axis or a short axis of a medical device within a body vessel. For instance, radiopaque material may be attached to a tubular drainage stent or woven into portions of the valve member material.

Methods of Manufacture

The medical devices can be formed in any suitable manner that provides a structure having a sleeve attached to a drainage stent. The sleeve preferably comprises a expanded PTFE and a bioactive agent.

When the term “expanded” is used to describe PTFE, i.e. ePTFE, it is intended to describe PTFE which has been stretched, in accordance with techniques which increase the internodal distance and concomitantly porosity. The stretching may be in uni-axially, bi-axially, or multi-axially. The nodes are stretched apart by the stretched fibrils in the direction of the expansion. Methods of making conventional longitudinally expanded ePTFE are well known in the art.

In one aspect, a billet comprising a PTFE resin is mixed with a bioactive agent. A billet can have a solvent level of about 10 to 30% by weight, to yield an extrudate suitable for the stretching process. Moreover, it is desired that the preformed billet is extruded to a reduction ratio of about 200 to 1. An additional parameter which has a significant effect on the resulting extrudate property upon being stretched is the extrusion pressure. Suitable extrusion pressures to practice the present invention include pressures of about 5,000 PSI to about 10,000 PSI.

The extrudate can be stretched under conditions capable of yielding a layer which is uniform over a large portion of its length. Stretching conditions are given in terms of stretch rate and stretch ratio. Stretch rate refers to the percentage change in length of the extrudate per unit time. Preferably, the stretch rate may be about 7 to about 8 inches per second (about 17.7 to 20.3 cm per second). The percentage change is calculated with reference to the starting length of the extrudate. In contrast, stretch ratio is not time dependent but refers to the ratio of the final length of the stretched extrudate to that of the initial length of the unstretched extrudate. With respect to a bioactive-containing sleeve, the stretch ratio can be about 2.5 to 1. Moreover, stretching is preferably conducted at a temperature of about 250° C. and the extrudate can be placed in tension during the stretching process.

An ePTFE sleeve can have enhanced axial elongation and radial expansion properties of up to about 600% or more by linear dimension. The physically modified ePTFE tubular structure is able to be elongated or expanded and then returned to its original state without an elastic force existing therewith. Additional details of physically-modified ePTFE and methods for making the same can be found in commonly assigned Application Title “ePTFE Graft With Axial Elongation Properties”, assigned U.S. application Ser. No. 09/898,418, filed on Jul. 3, 2001, published on Jan. 9, 2003 as U.S. Application Publication No. 2003-0009210A1, the contents of which are incorporated by reference herein in its entirety. Preferably, the sleeve is formed from ePTFE having pores of an internodal distance from about 5 to about 10 microns. After the extrudate sleeve has been stretched, the sleeve can be sintered by heating it above its crystalline melting point while under tension. This allows the microstructure of the material to be set properly.

Optionally, the ePTFE tube can be coated with an adhesive solution of from 1%-15% CORETHANE®, in Dimethylacetamide (DMAC). The coated ePTFE tubular structure is then placed in an oven heated in a range from 18° C. to 150° C. for 5 minutes to overnight to dry off the solution. The coating and drying process can be repeated multiple times to add more adhesive to the ePTFE tubular structure. Once dried, the ePTFE tubular sleeve structure may be longitudinally compressed in the axial direction to enhance the longitudinal stretch properties of the resulting sleeve. Longitudinal compression is performed in the axial direction to between 1% to 85% of its length to relax the fibrils of the ePTFE. Longitudinal expansion and compression may be balanced to achieve the desired properties. The longitudinal compression process can be performed either by manual compression or by thermal compression.

Optionally, the sleeve material can be formed from two or more layers of ePTFE bonded together to form a composite sleeve structure. The expansion and sintering of an outer sleeve layer over an inner sleeve tube serves to adherently bond the interface between two tubes, resulting in a single composite structure. A composite ePTFE sleeve structure may be formed by expanding a thin wall PTFE inner tube at a relatively high degree of elongation, on the order of approximately between 400 and 2,000% elongation and preferably from about between 500% and 600%. An inner tube is expanded over a cylindrical mandrel, such as a stainless steel mandrel at a temperature of between room temperature and 645° F., preferably about 500° F. The inner tube is preferably, but not necessarily fully sintered after expansion. Sintering is typically accomplished at a temperature of between 645° F. and 800° F., preferably at about 660° F. and for a time of between about 5 minutes to 30 minutes, preferably about 15 minutes. The combination of the inner ePTFE tube over the mandrel is then employed as a substrate over which a second layer. The interior diameter of the second tube is selected so that it may be easily but tightly disposed over the outside diameter of the inner tube. The composite structure formed between the two tubes is then sintered at preferably similar parameters. A bioactive agent can be incorporated in one or more layers of the multilayer structure.

Biodeposition-Reducing Bioactive Agents

Preferably, the sleeve comprises a bioactive agent selected to reduce or eliminate the deposition of sludge on the sleeve or within the drainage lumen of the drainage stent. The bioactive agent preferably includes one or more antimicrobial agents, antibiotic agents and antifungal agents.

One or more biodeposition-reducing bioactive materials can be incorporated in or coated on a sleeve by any suitable method. In one aspect, a dry, finely subdivided bioactive agent may be blended with the wet or fluid ePTFE material used to form the sleeve before the ePTFE solidifies. Alternatively, air pressure or other suitable means may be employed to disperse the bioactive agent substantially evenly within the pores of the solidified ePTFE. In situations where the bioactive agent is insoluble in the wet or fluid ePTFE material, the bioactive agent may be finely subdivided as by grinding with a mortar and pestle. Preferably, the bioactive agent is micronized, e.g., a product wherein some or all particles are the size of about 5 microns or less. The finely subdivided bioactive agent can then be distributed desirably substantially evenly throughout the bulk of the wet or fluid ePTFE layer before cross-linking or cure solidifies the layer. Alternatively, a bioactive agent can be incorporated into the ePTFE sleeve in the following manner: mixing a crystalline, particulate material (e.g., salt or sugar that is not soluble in a solvent used to form the extrudate) into an extrudate used to make the ePTFE sleeve; casting the extrudate solution with particulate material; and then applying a second solvent, such as water, to dissolve and remove the particulate material, thereby leaving a porous ePTFE. The ePTFE may then be placed into a solution containing a bioactive agent in order to fill the pores. Preferably, a vacuum would be pulled on the ePTFE to insure that the bioactive agent applied to it is received into the pores. Alternatively, the drug may be coated on the outside surface of the ePTFE. The drug may be applied to the outside surface of the ePTFE such as by dipping, spraying, or painting.

The bioactive agent may include antimicrobial or antibiotic agents. Suitable antibiotic bioactive agents include ciprofloxacin, vancomycin, doxycycline, amoxicillin, metronidazole, norfloxacin (optionally in combination with ursodeoxycholic acid), ciftazidime, and cefoxitin. Bactericidal nitrofuran compounds, such as those described by U.S. Pat. No. 5,599,321 (Conway et al.), incorporated herein by reference, can also be used as a bioactive agent. Preferred nitrofuran bioactive agents include nitrofurantoin, nitrofurazone, nidroxyzone, nifuradene, furazolidone, furaltidone, nifuroxime, nihydrazone, nitrovin, nifurpirinol, nifurprazine, nifuraldezone, nifuratel, nifuroxazide, urfadyn, nifurtimox, triafur, nifurtoinol, nifurzide, nifurfoline, nifuroquine, and derivatives of the same, and other like nitrofurans which are both soluble in water and possess antibacterial activity. References to each of the above cited nitrofuran compounds may be found in the Merck Index, specifically the ninth edition (1976) and the eleventh edition (1989) thereof, published by Merck & Co., Inc., Rahway, N.J., the disclosures of which are each incorporated herein by reference. Antibiotic agents also include cephalosporins, clindamycin, chloramphenicol, carbapenems, penicillins, monobactams, quinolones, tetracycline, macrolides, sulfa antibiotics, trimethoprim, fusidic acid and aminoglycosides. Antifungal agents include amphotericin B, azoles, flucytosine, cilofungin and nikkomycin Z.

Other suitable bioactive agents include bactericidal agents that inhibit bacterial DNA-dependent RNA polymerase activity such as rifampin, and antibiotic agents derived from tetracycline that inhibits protein synthesis such as minocycline, and agents that inhibit bacterial protein and nucleic acid synthesis, such as novobiocin. The bioactive agent can also be a combination of bioactive agents, such as those discussed in U.S. Pat. No. 5,217,493 (Raad et al.). Rifampin is a semisynthetic derivative of rifamycin B, a macrocyclic antibiotic compound produced by the mold Streptomyces mediterranic. Rifampin is available in the United States from Merrill Dow Pharmaceuticals, Cincinnati, Ohio. Minocycline is a semisynthetic antibiotic derived from tetracycline. It is primarily bacteriostatic and exerts its antimicrobial effect by inhibiting protein synthesis. Minocycline is commercially available as the hydrochloride salt which occurs as a yellow, crystalline powder and is soluble in water and slightly soluble in alcohol and is available from Lederle Laboratories Division, American Cyanamid Company, Pearl River, N.Y. Novobiocin is an antibiotic obtained from cultures of Streptomyces niveus or S. spheroides. Novobiocin is usually bacteriostatic in action and appears to interfere with bacterial cell wall synthesis and inhibits bacterial protein and nucleic acid synthesis. The drug also appears to affect stability of the cell membrane by complexing with magnesium. Novobiocin is available from The Upjohn Company, Kalamazoo, Mich.

The sleeve 13 can also comprise one or more antimicrobial agents. The term “antimicrobial” refers to inhibition of, prevention of or protection against microorganisms such as, bacteria, microbes, fungi, viruses, spores, yeasts, molds and others generally associated with infections such as those contracted from the use of the medical articles described here. The antimicrobial agents include antiseptic agents selected from the group consisting of silver, chlorhexidine, triclosan, iodine, benzalkonium chloride and other like agents. Examples of suitable antimicrobial materials also include nanosize particles of metallic silver or an alloy of silver containing about 2.5 wt % copper (hereinafter referred to as “silver-copper”), salts such as silver citrate, silver acetate, silver benzoate, bismuth pyrithione, zinc pyrithione, zinc percarbonates, zinc perborates, bismuth salts, various food preservatives such as methyl, ethyl, propyl, butyl, and octyl benzoic acid esters (generally referred to as parabens), citric acid, benzalkonium chloride (BZC), rifamycin and sodium percarbonate.

Optionally, materials with antimicrobial properties can be mixed with or applied to the surface of the sleeve 13. One example of a suitable antimicrobial material is described in published U.S. patent application US2005/0008763A1 (filed Sep. 23, 2003 by Schachter), incorporated herein by reference. The sleeve 13 can be combined with a siloxane binder and divalent metallic (M²⁺) ions, such as, for example, Cu²⁺, Zn²⁺, Ca²⁺, Co²⁺, and Mn²⁺. Upon curing, the siloxane binder can form a silsesquioxane, e.g., methyl silane sesquioxide or CH₃SiO_{3/2}. The siloxane oligomeric binder can be synthesized, for example by hydrolysis of precursors such as, for instance, monomethylalkoxysilane, e.g., methyltrimethoxysilane (CH₃Si(OCH₃)₃) to form a partial condensate of methyl trisilanol. The monomethylalkoxysilane also can be provided in a mixture with copolymerizable silane monomer(s). A copolymer may be formed from cohydrolyzed silanol, RSi(OH)₃, of which methyl trisilanol comprises at least about 70% by weight, preferably at least about 75% by weight, and wherein R is a non-reactive organic moiety, such as, for example, e.g., lower alkyl, e.g., C₁-C₆ alkyl, especially C₁-C₃ alkyl, e.g., methyl, ethyl or n- or iso-propyl, vinyl, 3,3,3-trifluoropropyl, γ-glycidyloxypropy, γ-methacryloxypropyl, and phenyl. When only methyl silanol (from methyl trialkoxysilane) is used, the amount of metal cation, (M²⁺) added can be based on the amount of silanol. When mixtures of silanol are used the molar silane sesquioxide equivalent of the remaining silane mixture can be converted to the molar equivalent of methyl silane sesquioxide. In one example, the composition includes, on a weight basis of the total composition, from about 28% to 71%, preferably from about 31% to 71% silanol (of which at least about 70% is methylsilanol), from about 29% to about 39% water, from 0 to about 31%, preferably from about 15 to about 30%, isopropanol or other volatile organic solvent, and an M²⁺ ion or a mixture of such M²⁺ ions, within the range of from about 0.5 to 3 millimoles (gram x millimoles), preferably about 1.2 to 2.4 millimoles, per molar equivalent of the partial condensate calculated as methyl silane sesquioxide. The pH of the mixture is adjusted to mildly to slightly acidic, such as between 2.5 and 6.2, preferably 2.8 to 6.0, more preferably 3.0 to 6.0. More particularly, the aqueous coating composition can include a dispersion of divalent metal cations (such as Ca²⁺, Mn²⁺, Cu²⁺, and Zn²⁺) in a solution of water/lower aliphatic alcohol of the partial condensate of at least one silanol of the formula RSi(OH)₃ in which R is a radical selected from the group consisting of lower alkyl, vinyl, phenyl, 3,3,3-trifluoropropyl, γ-glycidyloxypropyl and γ-methacryloxypropyl, at least about 70 weight percent of the silanol being CH₃Si(OH)₃, acid in an amount sufficient to provide a pH in the range of from about 2.5 to about 6.2, and said divalent cations in an amount of from about 1.2 millimoles to about 2.4 millimoles per molar equivalent of the partial condensate, calculated as methyl silane sesquioxide.

Optionally, the bioactive agent or drug may be encapsulated in microparticles, such as microspheres, microfibers or microfibrils, which can then be incorporated into or on the ePTFE sleeve. Various methods are known for encapsulating drugs within microparticles or microfibers (see Patrick B. Deasy, Microencapsulation and Related Drug Processes, Marel Dekker, Inc., New York, 1984). For example, a suitable microsphere for incorporation would have a diameter of about 10 microns or less. The microsphere could be contained within the mesh of fine fibrils connecting the matrix of nodes in the ePTFE sleeve. The microparticles containing the drug may be incorporated within a zone by adhesively positioning them onto the ePTFE material or by mixing the microparticles with a fluid or gel and flowing them into the ePTFE sleeve. The fluid or gel mixed with the microparticles could, for example, be a carrier agent designed to improve the cellular uptake of the bioactive agent incorporated into the ePTFE sleeve. Moreover, it is well within the contemplation of the present invention that carrier agents, which can include hyaluronic acid, may be incorporated within each of the embodiments of the present invention so as to enhance cellular uptake of the bioactive agent or agents associated with the device. The microparticles embedded in the ePTFE sleeve may have a polymeric wall surrounding the drug or a matrix containing the drug and optional carrier agents. Moreover, microfibers or microfibrils, which may be drug loaded by extrusion, can be adhesively layered or woven into the ePTFE.

Methods of Delivery and Treatment

A drainage stent can be delivered to a point of treatment within a body vessel in any suitable manner. Preferably, the drainage stent is delivered percutaneously. For example, a biliary stent can be inserted into a biliary lumen in one of several ways: by inserting a needle through the abdominal wall and through the liver (a percutaneous transhepatic cholangiogram or “PTC”), by cannulating the bile duct through an endoscope inserted through the mouth, stomach, and duodenum (an endoscopic retrograde cholangiogram or “ERCP”), or by direct incision during a surgical procedure. A preinsertion examination, PTC, ERCP, or direct visualization at the time of surgery may be performed to determine the appropriate position for stent insertion. A guidewire can then be advanced through the lesion, a delivery catheter is passed over the guidewire to allow the stent to be inserted. In general, plastic stents are placed using a pusher tube over a guidewire with or without a guiding catheter. Any suitable guidewire may be used for delivery of the device, such as a 0.035 inch wire guide for stent placement (such as the FUSION short guide wire or long guide wire systems, available from Cook Endoscopy, Winston-Salem, N.C.), which may be used in combination with an Intra Ductal Exchange (IDE) port. Delivery systems are now available for plastic stents that combine the guiding and pusher catheters (OASIS, Cook Endoscopy Inc., Winston-Salem, N.C.). Optionally, the diameter of the pusher catheter can be reduced at the distal end, which is positioned behind the drainage stent, permitting the sleeve to enclose the pusher.

The biliary stent may be placed in the biliary duct either by the conventional pushing technique or by mounting it on a rotatable delivery catheter having a biliary stent engaging member engageable with one end of the stent. Typically, when the diagnostic exam is a PTC, a guidewire and delivery catheter may be inserted via the abdominal wall. If the original exam was an ERCP, the biliary stent may be placed via the mouth. The biliary stent may then positioned under radiologic, endoscopic, or direct visual control at a point of treatment, such as across the narrowing in the bile duct. The billiary stent may be released using the conventional pushing technique. The delivery catheter may then be removed, leaving the biliary stent to hold the bile duct open. A further cholangiogram may be performed to confirm that the biliary stent is appropriately positioned. Alternatively, other drainage stents can also be delivered to any suitable body vessel, such as a vein, artery, urethra, ureteral passage or portion of the alimentary canal.

The invention includes other embodiments within the scope of the claims, and variations of all embodiments, and is limited only by the claims made by the Applicants. Additional understanding of the invention can be obtained by referencing the detailed description of embodiments of the invention, below, and the appended drawings. It is to be understood that the above described anti-reflux biliary prostheses 10 is merely an illustrative embodiment of this invention. The present invention can also include other devices and methods for manufacturing and using them may be devised by those skilled in the art without departing from the spirit and scope of the invention. The invention also includes embodiments both comprising and consisting of disclosed parts. For example, it is contemplated that the entire tubular drainage stent can be coated with the sleeve material. Furthermore, the sleeve material extending from the distal end of the tubular member can be formed with different material from that covering the tubular drainage stent. It is also contemplated that the material of the stents can be formed of other materials such as nickel titanium alloys commercially known as nitinol, spring steel, and any other spring-like material formed to assume a flexible self-expanding zig-zag stent configuration.

We claim:

1. A medical device for placement in a patient comprising: a tubular member adapted for placement in a bodily passageway, the tubular member having a drainage lumen extending longitudinally through the tubular member, and a sleeve comprising a flexible material and a biodeposition-reducing bioactive agent attached to the tubular member, the sleeve defining a collapsible lumen in fluid flow communication with the drainage lumen of the drainage stent.

2. The medical device of claim 1, wherein the sleeve is moveable in response to a fluid applying a first pressure in a first direction passing the fluid through the lumen thereof, the sleeve collapsible to at least substantially close the lumen in response to either a fluid applying a second pressure in a second direction or the absence of the fluid applying a first pressure in a first direction.

3. The medical device of claim 1, wherein the drainage lumen of the drainage stent extends longitudinally from an inlet to an outlet, and the sleeve extends longitudinally from the outlet of the drainage stent.

4. The medical device of claim 1, wherein the sleeve is positioned entirely within the drainage lumen of the tubular member.

5. The medical device of claim 1, wherein the biodeposition-reducing bioactive agent is selected from the group consisting of: an antimicrobial agent and an antibiotic agent.

6. The medical device of claim 1, wherein the biodeposition-reducing bioactive agent comprises a material selected from the group consisting of: cephalosporins, clindamycin, chloramphenicol, carbapenems, penicillins, monobactams, quinolones, tetracycline, macrolides, sulfa antibiotics, trimethoprim, fusidic acid, aminoglycosides, vancomycin, chlorhexidine, triclosan, iodine, ampicillin, rifampin, minocycline, novobiocin, ciprofloxacin, doxycycline, amoxicillin, metronidazole, norfloxacin, ciftazidime, cefoxitin, nitrofurantoin, nitrofurazone, nidroxyzone, nifuradene, furazolidone, furaltidone, nifuroxime, nihydrazone, nitrovin, nifurpirinol, nifurprazine, nifuraldezone, nifuratel, nifuroxazide, urfadyn, nifurtimox, triafur, nifurtoinol, nifurzide, nifurfoline, nifuroquine, metallic silver, an alloy of silver containing about 2.5 wt % copper, silver citrate, silver acetate, silver benzoate, bismuth pyrithione, zinc pyrithione, zinc percarbonates, zinc perborates, bismuth salts, benzalkonium chloride (BZC), rifamycin and sodium percarbonate.

7. The medical device of claim 1, wherein sleeve comprises a material selected from the group consisting of: expanded polytetrafluoroethylene and polyurethane.

8. The medical device of claim 1, wherein the tubular member is drainage stent.

9. The medical device of claim 1, wherein the tubular member is a biliary stent further comprising an anchoring means for securing the drainage stent within a biliary duct.

10. The medical device of claim 9, wherein the collapsible lumen of the sleeve is positioned within the drainage lumen of the biliary stent or extends from the outlet of the biliary stent.

11. The medical device of claim 1, wherein the tubular member is a biliary stent; wherein sleeve comprises expanded polytetrafluoroethylene, the biliary stent comprises polyethylene, wherein the drainage lumen of the biliary stent extends longitudinally from an inlet to an outlet, wherein the sleeve extends longitudinally from the outlet of the drainage stent and wherein the biliary stent comprises a plurality of extending flaps positioned proximate the outlet or the inlet.

12. The medical device of claim 11, wherein the biodeposition-reducing bioactive agent is selected from the group consisting of: cephalosporins, clindamycin, chloramphenicol, carbapenems, penicillins, monobactams, quinolones, tetracycline, macrolides, sulfa antibiotics, trimethoprim, fusidic acid, aminoglycosides, vancomycin, chlorhexidine, triclosan, iodine, ampicillin, rifampin, minocycline, novobiocin, ciprofloxacin, doxycycline, amoxicillin, metronidazole, norfloxacin, ciftazidime, cefoxitin nitrofurantoin, nitrofurazone, nidroxyzone, nifuradene, furazolidone, furaltidone, nifuroxime, nihydrazone, nitrovin, nifurpirinol, nifurprazine, nifuraldezone, nifuratel, nifuroxazide, urfadyn, nifurtimox, triafur, nifurtoinol, nifurzide, nifurfoline, nifuroquine, metallic silver, an alloy of silver containing about 2.5 wt % copper, silver citrate, silver acetate, silver benzoate, bismuth pyrithione, zinc pyrithione, zinc percarbonates, zinc perborates, bismuth salts, benzalkonium chloride (BZC), rifamycin and sodium percarbonate.

13. A drainage stent comprising: an elongated tubular member having an exterior surface and an interior surface defining a drainage lumen extending longitudinally from an inlet to an outlet, and a sleeve comprising a flexible material and a biodeposition-reducing bioactive agent, the sleeve disposed around the outlet of the tubular drainage stent; the sleeve extending from outlet of the tubular member and having a collapsible sleeve lumen extending longitudinally through the sleeve in fluid flow communication with the drainage lumen defined by the interior surface of the tubular member; the sleeve being adapted to open in response to a fluid applying a first pressure in a first direction passing the fluid through the drainage lumen through the sleeve lumen; and the sleeve further being adapted to collapse the sleeve lumen in response a fluid applying a second pressure in a second direction.

14. The drainage stent of claim 13, wherein the biodeposition-reducing bioactive agent is selected from the group consisting of: an antimicrobial agent and an antibiotic agent.

15. The drainage stent of claim 13, wherein the biodeposition-reducing bioactive agent comprises a compound selected from the group consisting of: cephalosporins, clindamycin, chloramphenicol, carbapenems, penicillins, monobactams, quinolones, tetracycline, macrolides, sulfa antibiotics, trimethoprim, fusidic acid, aminoglycosides, vancomycin, chlorhexidine, triclosan, iodine, ampicillin, rifampin, minocycline, novobiocin, ciprofloxacin, doxycycline, amoxicillin, metronidazole, norfloxacin, ciftazidime, cefoxitin, nitrofurantoin, nitrofurazone, nidroxyzone, nifuradene, furazolidone, furaltidone, nifuroxime, nihydrazone, nitrovin, nifurpirinol, nifurprazine, nifuraldezone, nifuratel, nifuroxazide, urfadyn, nifurtimox, triafur, nifurtoinol, nifurzide, nifurfoline, nifuroquine, metallic silver, an alloy of silver containing about 2.5 wt % copper, silver citrate, silver acetate, silver benzoate, bismuth pyrithione, zinc pyrithione, zinc percarbonates, zinc perborates, bismuth salts, benzalkonium chloride (BZC), rifamycin and sodium percarbonate.

16. The medical device of claim 13 configured as a drainage stent adapted for placement within a biliary or pancreatic duct, the drainage stent comprising polyethylene or polyurethane.

17. The drainage stent of claim 16, wherein the biodeposition-reducing bioactive agent comprises a compound selected from the group consisting of: cephalosporins, clindamycin, chloramphenicol, carbapenems, penicillins, monobactams, quinolones, tetracycline, macrolides, sulfa antibiotics, trimethoprim, fusidic acid, aminoglycosides, vancomycin, chlorhexidine, triclosan, iodine, ampicillin, rifampin, minocycline, novobiocin, ciprofloxacin, doxycycline, amoxicillin, metronidazole, norfloxacin, ciftazidime, cefoxitin, nitrofurantoin, nitrofurazone, nidroxyzone, nifuradene, furazolidone, furaltidone, nifuroxime, nihydrazone, nitrovin, nifurpirinol, nifurprazine, nifuraldezone, nifuratel, nifuroxazide, urfadyn, nifurtimox, triafur, nifurtoinol, nifurzide, nifurfoline, nifuroquine, metallic silver, an alloy of silver containing about 2.5 wt % copper, silver citrate, silver acetate, silver benzoate, bismuth pyrithione, zinc pyrithione, zinc percarbonates, zinc perborates, bismuth salts, benzalkonium chloride (BZC), rifamycin and sodium percarbonate.

18. A method of treating a condition associated with reduced fluid flow through a body vessel, the method comprising the steps of:
providing a drainage stent comprising a tubular member having an exterior surface and an interior surface defining a drainage lumen extending along the longitudinal axis of the tubular member from an inlet to an outlet, and a sleeve extending longitudinally from the outlet, the sleeve comprising a biodeposition-reducing bioactive agent and defining a collapsible lumen in fluid flow communication with the drainage lumen defined by the interior surface of the tubular member; and
implanting the drainage stent within a body vessel.

19. The method of claim 18, wherein the condition is selected from the group consisting of: obstructive jaundice, postoperative biliary stricture, primary sclerosing cholangitis and chronic pancreatitis.

20. The method of claim 18, wherein the tubular member comprises polyethylene and the sleeve comprises expanded polytetrafluoroethylene; and wherein the biodeposition-reducing bioactive agent comprises a compound selected from the group consisting of: cephalosporins, clindamycin, chloramphenicol, carbapenems, penicillins, monobactams, quinolones, tetracycline, macrolides, sulfa antibiotics, trimethoprim, fusidic acid, aminoglycosides, vancomycin, chlorhexidine, triclosan, iodine, ampicillin, rifampin, minocycline, novobiocin, ciprofloxacin, doxycycline, amoxicillin, metronidazole, norfloxacin, ciftazidime, cefoxitin, nitrofurantoin, nitrofurazone, nidroxyzone, nifuradene, furazolidone, furaltidone, nifuroxime, nihydrazone, nitrovin, nifurpirinol, nifurprazine, nifuraldezone, nifuratel, nifuroxazide, urfadyn, nifurtimox, triafur, nifurtoinol, nifurzide, nifurfoline, nifuroquine, metallic silver, an alloy of silver containing about 2.5 wt % copper, silver citrate, silver acetate, silver benzoate, bismuth pyrithione, zinc pyrithione, zinc percarbonates, zinc perborates, bismuth salts, benzalkonium chloride (BZC), rifamycin and sodium percarbonate.
